(19) United States
(12) Patent Application Publication
(10) Pub. No.: US 2013/0274872 A1
(43) Pub. Date: Oct. 17, 2013

(54) RESIZABLE VALVE BASE FOR CARDIOVASCULAR VALVE ASSEMBLY

(71) Applicant: Ivan Vesely, Larkspur, CO (US)

(72) Inventor: Ivan Vesely, Larkspur, CO (US)

(73) Assignee: ValveXchange Inc.

(21) Appl. No.: 13/995,559

(22) PCT Filed: Jan. 5, 2012
(86) PCT No.: PCT/US12/20298

§ 371 Date: Jun. 19, 2013

Related U.S. Application Data
(60) Provisional application No. 61/430,339, filed on Jan. 6, 2011.

(51) Int. Cl. A61F 2/24

(52) U.S. Cl. CPC A61F 2/2403
USPC 623/2.17

(57) ABSTRACT

A resizable valve base for a cardiovascular valve assembly that is comprised of the resizable valve base and a valve member that is detachably coupled thereto. The resizable valve base has adjustable dimensions to allow use with both small and large size valve members. The valve base comprises a frame comprised of a plurality of frame sections, and each frame section includes a mounting portion and a pair of arcuate portions extending from opposite sides of the mounting portion. The valve base also includes a plurality of joining elements for joining together the frame sections to define a generally cylindrical opening having a diameter, wherein said plurality of joining elements allow adjacent frame sections to move towards or away from each other in order to modify the diameter of the opening, thereby moving the valve base between a collapsed position and an expanded position.

### RELATED APPLICATIONS

This application claims the benefit of U.S. Provisional Patent Application No. 61/430,339 (filed on Jan. 6, 2011), hereby fully incorporated herein by reference.

### FIELD OF THE INVENTION

The present invention relates generally to a cardiovascular valve assembly, and more particularly to a resizable valve base for a cardiovascular valve assembly that is comprised of the resizable valve base and an exchangeable valve member that is detachably coupled thereto.

### BACKGROUND OF THE INVENTION

The concept of a bioprosthetic cardiovascular valve assembly comprised of an exchangeable valve member detachably coupled to a valve base (also referred to as a “base member” or “docking station”) is known in the prior art. For example, see U.S. Patent Application No. 13/063,218 (published as US 2011/0167603), which is fully incorporated herein by reference. Existing exchangeable valve members include a rigid support frame having flexible stent posts. A leaflet set (e.g., animal tissue leaflets) is supported by the frame. A biocompatible cloth (e.g., Dacron®) covers components of the valve assembly.

The valve base is permanently installed in the patient, while the valve member is detachably coupled to the valve base. When the leaflet set of the valve member wears out, the existing valve member is decoupled from the valve base and replaced with a new valve member using a variety of surgical or interventional approaches.

If a conventional valve assembly is applied to a young pediatric patient (e.g., a young teenager) that is expected to grow, a conventional adult-size valve assembly (e.g., 21 mm diameter) cannot be used. Instead, such patients are candidates for a much smaller pediatric-size valve assembly (e.g., 17 mm diameter).

However, tissue leaflets are known to wear out very quickly (e.g., about 5 years) in such pediatric patients. Therefore, it would be advantageous if at the time the valve member requires replacement due to worn leaflets, the pediatric-size valve base could be upsized to an adult-size valve base that accepts an adult-size valve member. The patient is likely to be in their early 20s at the time of such valve member replacement, and thus would be a candidate for an adult-size valve member.

Another problem encountered with existing valves is that it can be difficult to select a valve that is properly sized to fit into the aortic annulus of a patient. The aortic annulus is defined as the constriction between the ventricle and the Sinuses of Valsalva. Currently, a properly sized valve is selected by inserting several different sizers into the aortic annulus and, by trial and error, determining which sizer fits that patient best. Thereafter, a valve of the determined size is implanted into the patient. A resizable valve base would simplify the process for installing a valve of the proper size.

The present invention addresses the drawbacks of existing cardiovascular valve assemblies by providing a resizable valve base that is adaptable to couple with valve members of different sizes.

### SUMMARY OF THE INVENTION

In accordance with the present invention, there is provided a resizable valve base for a cardiovascular valve assembly that is comprised of the resizable valve base and a valve member that is detachably mountable thereto.

An advantage of the present invention is the provision of a valve base that can be easily resized to couple with valve members of different sizes.

Another advantage of the present invention is the provision of a valve base that is adaptable to couple with both child-size and adult-size valve members.

Still another advantage of the present invention is the provision of a valve base that can be upsized after installation in a patient, thereby allowing both small and large diameter valve members to be coupled thereto.

Still another advantage of the present invention is the provision of a valve base that can be upsized during a valve member exchange procedure several years after installation in a patient, thereby accommodating an aorta that has grown to a larger size.

Still another advantage of the present invention is the provision of a valve base that simplifies the process for determining a properly sized valve for a patient.

Still another advantage of the present invention is the provision of a valve base that is expandable while located in the aortic annulus during implantation of the valve assembly, thereby enabling installation of the largest suitable valve member.

Yet another advantage of the present invention is the provision of a valve base that is expandable while located in the aortic annulus during implantation, and anchorable within the aortic annulus without the use of securing sutures.

These and other advantages will become apparent from the following description taken together with the accompanying drawings and the appended claims.

### BRIEF DESCRIPTION OF THE DRAWINGS

The invention may take physical form in certain parts and arrangement of parts, an embodiment of which will be described in detail in the specification and illustrated in the accompanying drawings which form a part hereof, and wherein:

FIG. 1 is an exploded perspective view of a prior art cardiovascular valve assembly, the valve assembly including an exchangeable valve member and a valve base;

FIG. 2 is a perspective view of the valve assembly of FIG. 1, wherein the valve member is shown coupled to the valve base;

FIG. 3 is a perspective view of the prior art valve assembly shown in FIG. 2 with a fabric cover covering components of the assembly and a sewing ring or cuff for attachment of the valve base to the patient;

FIG. 4 is a perspective view of a size-adjustable valve base according to a first embodiment of the present invention, wherein the size-adjustable valve base is illustrated in an expanded position;

FIG. 5 is a cross-sectional view of the size-adjustable valve base, taken along lines 5-5 of FIG. 4;

FIG. 6 is a perspective view of the size-adjustable valve base shown in FIG. 4, wherein the valve base is illustrated in a collapsed position (i.e., original non-expanded configuration);

FIG. 7 is a cross-sectional view of the size-adjustable valve base, taken along lines 7-7 of FIG. 6;

FIG. 8 is a partially-sectioned side elevational view of the size-adjustable valve base as shown in FIG. 4;

FIG. 9 is a partial perspective view of the size-adjustable valve base as shown in FIG. 4;

FIG. 10 is a perspective view of a size-adjustable valve base according to an alternative embodiment of the present invention, wherein the size-adjustable valve base is illustrated in a collapsed position;

FIG. 11 is a perspective view of the size-adjustable valve base of FIG. 10, wherein the valve base is illustrated in an expanded position;

FIG. 12 is a cross-sectional view showing a valve base installed within an aortic annulus using a sewing cuff sutured thereto;

FIG. 13 is a cross-sectional view showing a modified valve base installed within an aortic annulus using upper and lower sewing cuffs to capture the aortic annulus;

FIG. 14 is a cross-sectional view showing a modified valve base installed within an aortic annulus using a sewing cuff extending within and below the annulus; and

FIG. 15 is a cross-sectional view showing a valve base installed within an aortic annulus using a sewing cuff extending within and above the annulus.

### DETAILED DESCRIPTION OF THE INVENTION

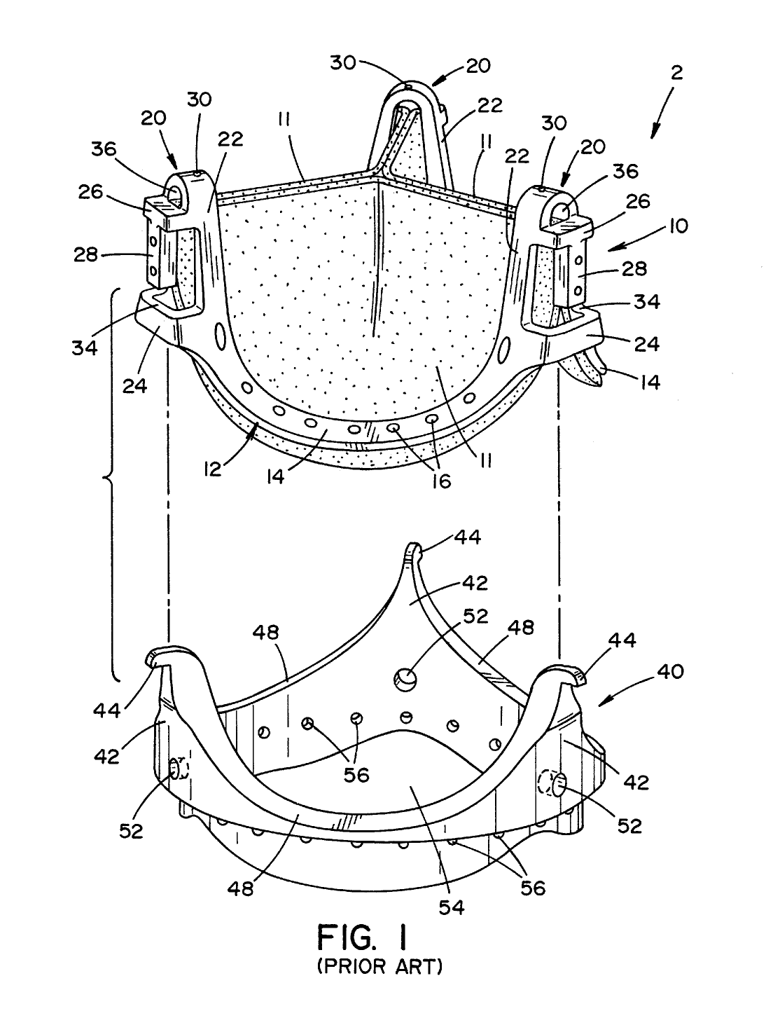

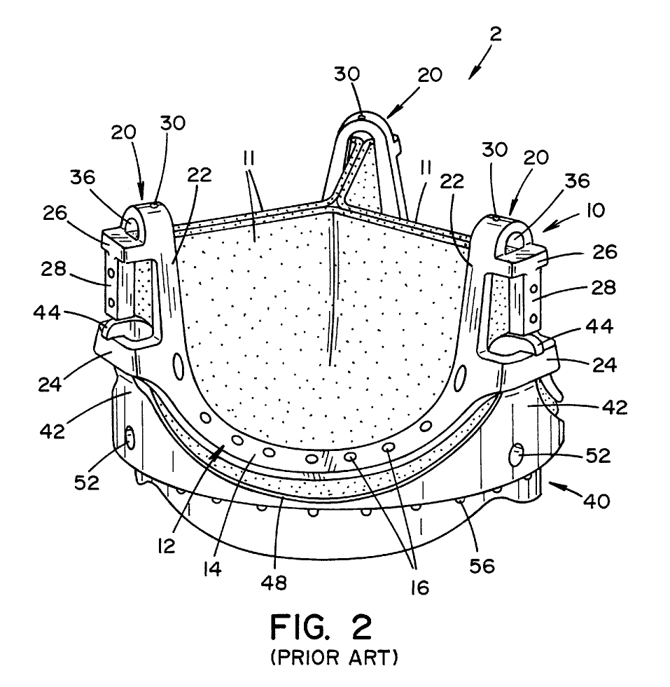

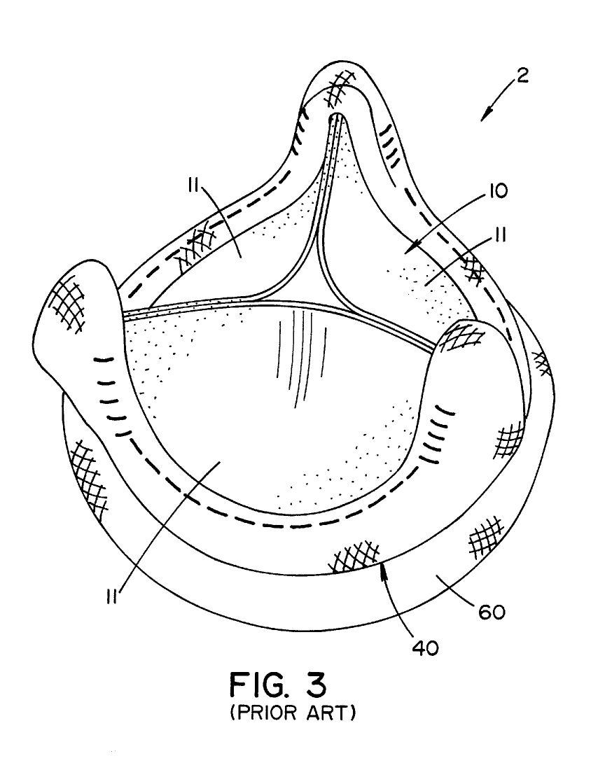

Referring now to the drawings wherein the showings are for the purposes of illustrating embodiments of the invention only and not for the purposes of limiting same, FIGS. 1-3 illustrate a cardiovascular valve assembly 2 known in the prior art. FIG. 1 shows an exploded perspective view of cardiovascular valve assembly 2. Valve assembly 2 is comprised of a valve base 40 and a valve member 10 that is detachably coupled to base 40. FIG. 2 shows valve member 10 coupled to base 40. In the illustrated embodiment, valve member 10 is a bioprosthetic valve. FIG. 3 also shows valve member 10 coupled to base 40 and illustrates a cloth cover that covers the components of valve assembly 2 and a sewing cuff 60 that is attached to base 40.

Base 40 is generally comprised of a plurality of mounting portions 42 and a plurality of arcuate sections 48 located between mounting portions 42. Mounting portions 42 function as stent posts of base 40. Each mounting portion 42 includes an outward extending protuberance or tab 44. Mounting portions 42 and arcuate sections 48 define a generally cylindrical recess 54. A plurality of recesses 52 may be formed in base 40 to facilitate engagement of base 40 by the use of tools. Recesses 52 may take the form of a hole or depression formed in base 40. Base 40 may be made of the same materials that are suitable for frame 12.

Sewing cuff 60 (see FIG. 3), made of Dacron® or other medical grade fabric, is sewn to the outer surface of base 40 using holes 56 located along the circumference of base 40. Sewing cuff 60 is used to attach base 40 to the tissue of the heart. Sewing cuff 60 may also include a sleeve section to provide further coverage of the outer surface of base 40.

Valve member 10 is generally comprised of a frame 12 and a plurality of valve leaflets 11 (i.e., a leaflet set) supported by frame 12. Frame 12 includes a plurality of ribbon sections 14 and coupling elements 20. Coupling elements 20 function as stent posts of valve member 10, and allow valve member 10 to be coupled and uncoupled from base 40, as will be described below.

Each coupling element 20 is comprised of a generally U-shaped portion 22 having lower and upper crossbars 24, 26 extending across U-shaped portion 22. Upper crossbar 26 is T-shaped and includes a downward extending finger 28. Finger 28 and lower crossbar 24 define a lower slot 34. Upper crossbar 26 and the top section of U-shaped portion 22 define an upper slot 36. A fabric cover (see FIG. 3) made of a medical grade cloth may be placed over each coupling element 20. In one embodiment of valve member 10, each coupling element 20 includes an opening 30 in generally U-shaped portion 22.

Each ribbon section 14 has a generally arcuate shape, and extends between coupling elements 20. Ribbon sections 14 have an arcuate shape that matches the profile of arcuate sections 48 of base 40, thereby forming a seal therebetween when valve member 10 is coupled to base 40. This seal prevents blood leakage between valve member 10 and base 40.

Frame 12 is preferably made of a flexible material having suitable elasticity to allow frame 12 to collapse into a tight bundle for convenient removal and exchange of valve member 10 through small incisions or a trocar, and to facilitate the engagement and disengagement of coupling elements 20 and mounting portions 42, as will be described below. In the illustrated embodiment, frame 12 is made of a medical grade polymer material, such as poly-ether-ether-ketone (PEEK), polyurethane or polycarbonate. However, frame 12 may alternatively be formed of a metal, including, but not limited to, Elgiloy, nitinol, stainless steel, platinum, gold, titanium, other biocompatible metals, and combinations thereof.

As indicated above, leaflets 11 are supported by frame 12. In this regard, leaflets 11 may be sewn to ribbon sections 14 using holes 16 formed along the length of ribbon sections 14. Alternatively, leaflets 11 may be attached to ribbon sections 14 by appropriate means, such as sutures, clips, staples or other fastening devices. Leaflets 11 may be made of suitable materials, including, but not limited to, bovine pericardium, equine pericardium, ovine pericardium, porcine aortic valve tissue, small intestinal submucosa (SIS), various biodegradable substrates for tissue engineered valves, and various relatively inert polymers, such as polyurethane.

Each pair of mounting portion 42/coupling element 20 provides a protuberance-slot mechanism. However, the configuration may be reversed, wherein each coupling element 20 provides a protuberance and each mounting portion 42 provides a slot. The entire protuberance-slot mechanism is covered by a fabric cover (see FIG. 3) that prevents fibrotic ingrowth. In FIG. 3, surfaces of valve assembly 2 are shown covered by a fabric cover (e.g., Dacron® or other medical grade fabric).

Valve member 10 is coupled and uncoupled to/from base 40 through engagement and disengagement of coupling elements 20 and mounting portions 42. Lower slot 34 of each coupling element 20 is dimensioned to receive a respective tab 44 of each mounting portion 42, thereby coupling valve member 10 to base 40. As best seen in FIG. 2, tab 44 is captured between the lower surface of finger 28 of upper crossbar 26 and the upper surface of lower crossbar 24. As discussed above, frame 12 is formed of an elastic material. Accordingly, frame 12 is dilated by outward deflection to disengage tab 44 of each mounting portion 42 from lower slot 34 of each coupling element 20. Consequently, valve member 10 is uncoupled from base 40. Coupling and uncoupling of valve member 10 to/from base 40 may be facilitated by use of a specially adapted tool set, such as described in U.S. Patent Application No. 13/063,218. Once valve member 10 is coupled to base 40, valve member 10 is secured such that it cannot unintentionally uncouple from base 40. In this respect, outward deflection of frame 12 is opposite to normal cardiac forces, thus providing secure engagement.

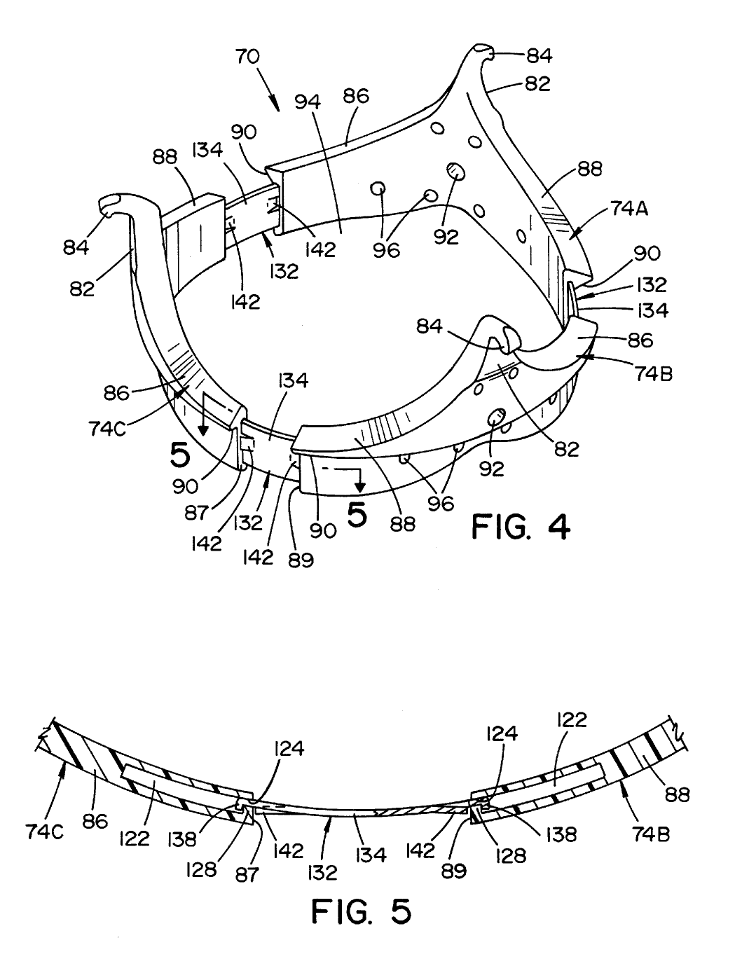

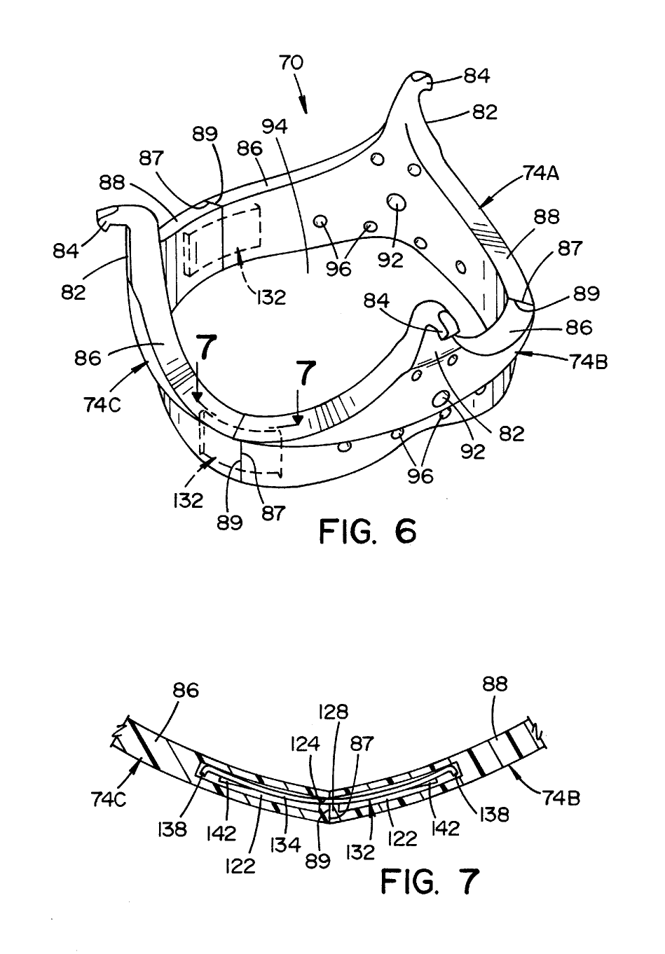

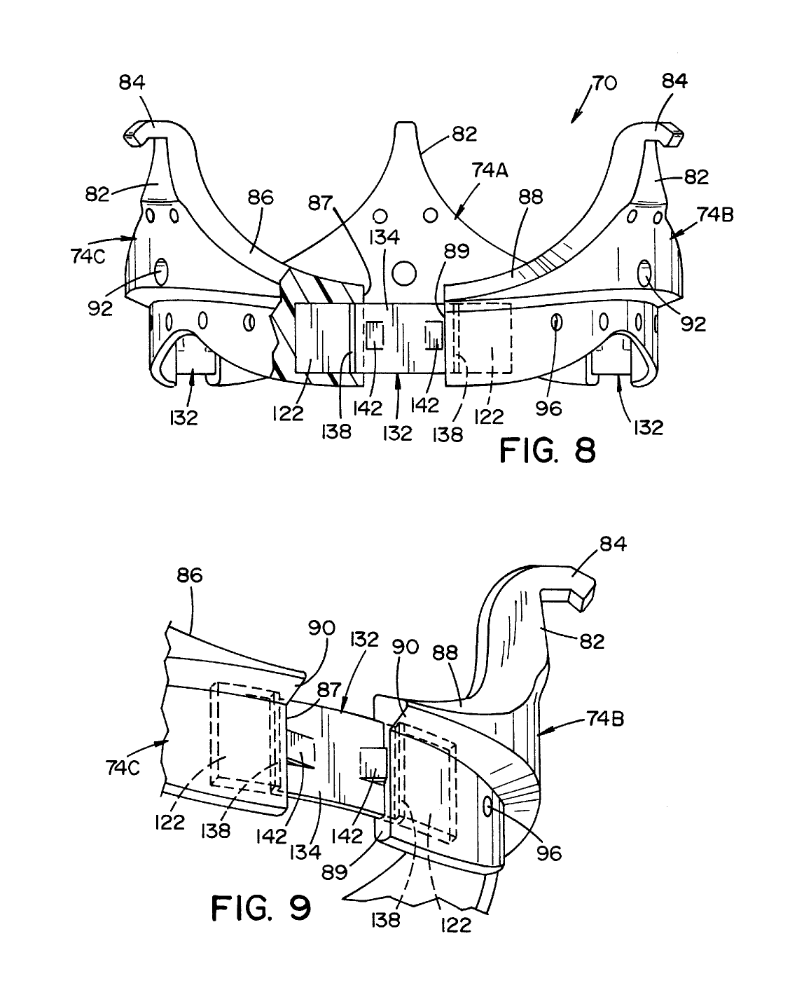

Referring now to FIGS. 4-9, there is shown a size-adjustable valve base 70 according to a first embodiment of the present invention. Base 70 is moveable between a collapsed position (FIGS. 6 and 7) and an expanded position (FIGS. 4-5 and 8-9) in order to accommodate valve members 10 of different sizes. For example, in the collapsed position, base 70 may be sized to couple with a valve member 10 having a diameter of 17 mm, whereas in the expanded position, base 70 may be sized to couple with a valve member 10 having a diameter of 21 mm. Operation of base 70 will be described in further detail below.

Base 70 is comprised of a frame that includes a plurality of C-shaped frame sections 74A, 74B, 74C and a plurality of joining elements 132 that connect together frame sections 74A, 74B and 74C. Frame sections 74A, 74B and 74C joined together by joining elements 132 define a generally cylindrical opening 94. The plurality of joining elements 132 allow adjacent frame sections 74A, 74B and 74C to move towards or away from each other in order to modify the diameter of opening 94, thereby moving the base between a collapsed position and an expanded position.

Each frame section 74A, 74B, 74C is generally comprised of a mounting portion 82 that functions as a stent post, and a pair of lateral arcuate portions 86 and 88 that extend from opposite sides of mounting portion 82. Each mounting portion 82 includes an outward extending protuberance or tab 84. Arcuate portions 86 and 88 have respective front faces 87 and 89. Each frame section 74A, 74B, 74C also has a flange 90. A plurality of recesses 92 may be formed in each frame section 74A, 74B, 74C to facilitate engagement of base 70 by specially adapted tools (not shown). Recesses 92 may take the form of a hole or depression.

A sewing cuff, such as shown in FIG. 3, may be sewn to the outer surface of base 70 using holes 96 located along the lower end of each frame section 74A, 74B and 74C and/or other holes (not shown) formed in base 70. The sewing cuff is used to permanently attach base 70 to the tissue of the heart. In FIG. 12, sewing cuff 60 is shown sutured to an aortic annulus, thereby installing base 70 therein. Sewing cuff 60 is sewn to base 70 such that at least a portion of sewing cuff 60 is located adjacent to flange 90. The sewing cuff may also include a sleeve section (not shown) to provide further coverage of the outer surface of base 70. It should be appreciated that the material for the sewing cuff is selected to provide elasticity such that the sewing cuff is suitably stretchable as base 70 is moved between a collapsed position and an expanded position. The stretchable sewing cuff is attached to base 70 such that expansion of the sewing cuff is generally uniform, and not only at locations where frame sections 74A, 74B, 74C slide apart to expand the diameter of base 70.

As shown in FIGS. 5 and 7, each arcuate portion 86 and 88 has an elongated inner channel 122 and a slot 124 in communication with inner channel 122. Channel 122 and slot 124 define an L-shaped wall 128. In the illustrated embodiment, joining element 132 takes the form of a curved band 134 having flange portions 138 at opposite ends thereof. Inner channel 122 is dimensioned to receive band 134 and allow band 134 to be slid into and out from inner channel 122 in order to adjust the diameter of cylindrical opening 94, and thereby couple with valve members 10 of different sizes.

L-shaped wall 128 prevents joining element 132 from separating from the associated frame sections by capturing flange portion 138 of band 134 (FIG. 5). In the illustrated embodiment, inner channel 122 is dimensioned such that flange portion 138 of band 134 makes a friction fit within inner channel 122. Band 134 may also include locking members in the form of flexible bent tabs 142. Tabs 142 are punched out of the sheet metal forming band 134 before band 134 is slid into inner channels 122. As band 134 slides out of inner channels 122 during expansion of base 70, tabs 142 flex outward thereby preventing band 134 from sliding back into inner channels 122. Accordingly, base 70 is prevented from returning to a collapsed position after expansion. It should be appreciated that additional tabs 142 may be provided to allow multiple expansion positions for base 70.

While FIGS. 5 and 7 show an embodiment wherein band 134 is slideable within inner channels 122 of both adjacent arcuate portions 86 and 88, it is also contemplated that band 134 may be fixed to one of the two arcuate portions 86 and 88. In this embodiment, band 134 is slidable within inner channel 122 of only one of the two arcuate portions 86, 88 (i.e., joining element 132 is movable relative to a first frame section and fixed relative to a second frame section).

Frame sections 74A, 74B, 74C and joining elements 132 may be made of a medical grade polymer material (such as, poly-ether-ether-ketone (PEEK), polyurethane or polycarbonate) or a metal (such as, Elgiloy, nitinol, stainless steel, platinum, gold, titanium, other biocompatible metals, and combinations thereof).

Operation of valve base 70 will now be described in detail. Base 70 is initially assembled in a collapsed position thus having its smallest diameter of cylindrical opening 94. The ends of each band 134 are inserted into inner channels 122 such that front faces 87, 89 of adjacent arcuate portions 86, 88 are moved toward each other. In the fully collapsed position, adjacent arcuate portions 86, 88 abut each other (see FIGS. 6 and 7). Therefore, frame sections 74A, 74B and 74C are located relative to each other such that base 70 is dimensioned to couple with a valve member 10 having a relatively small diameter (e.g., 17 mm).

To move base 70 to an expanded position and thereby increase the diameter of cylindrical opening 94, the ends of each band 134 are slid out of inner channels 122 such that front faces 87, 89 of adjacent arcuate portions 86, 88 are moved away from each other (see FIGS. 4 and 5). In the fully expanded position, flange portions 138 are captured by L-shaped wall 128, as seen in FIG. 5. Therefore, frame sections 74A, 74B and 74C are located relative to each other such that base 70 is dimensioned to couple to a valve member 10 having a relatively large diameter (e.g., 21 mm).

It should be appreciated that base 70 may be moved to a position intermediate to the fully collapsed and fully expanded positions in order to accommodate a valve member 10 having an intermediate diameter.

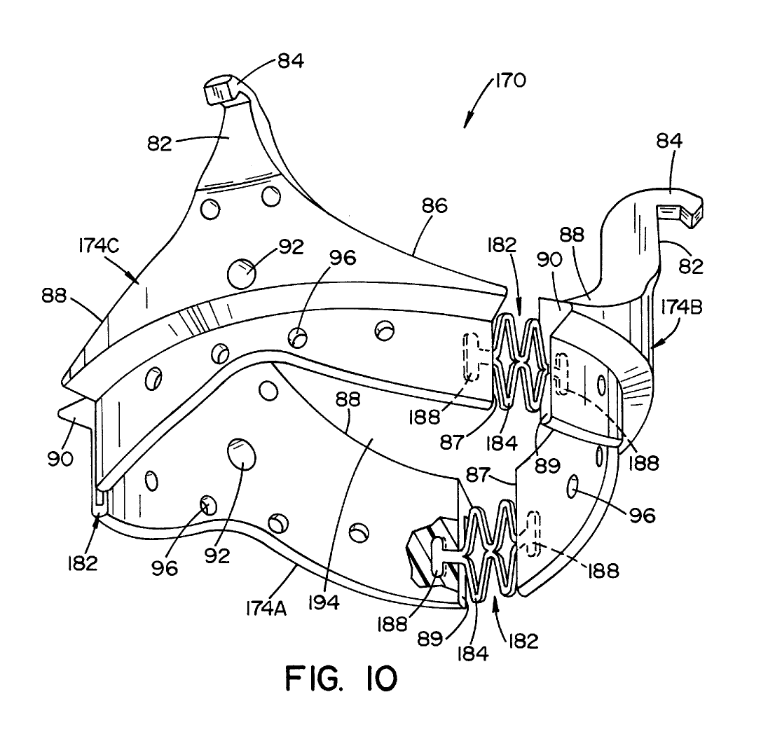

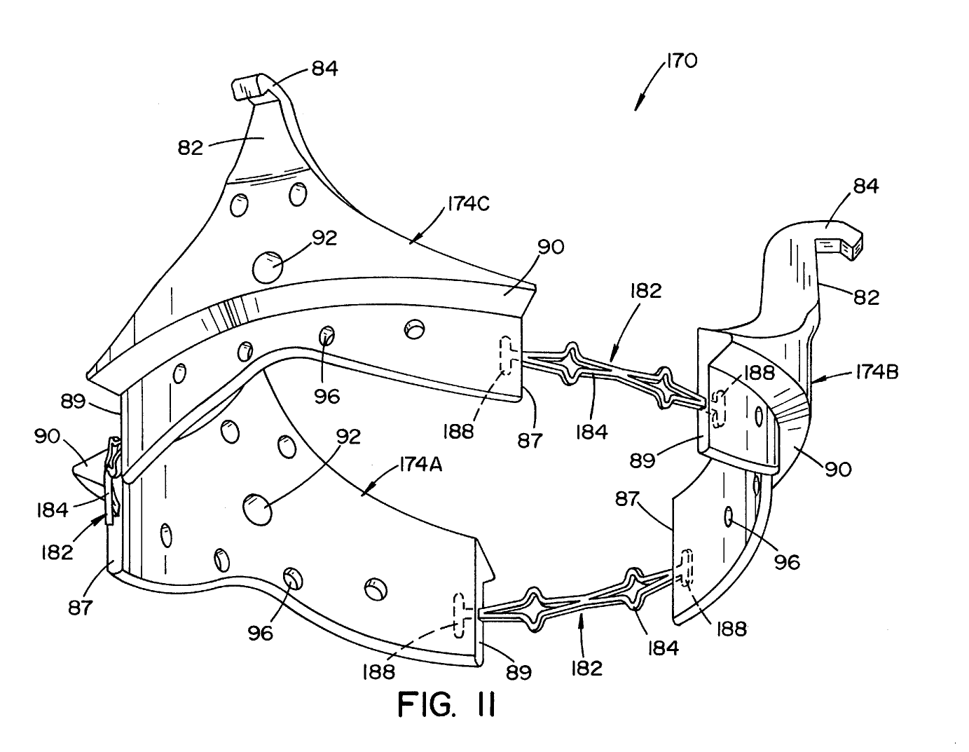

Referring now to FIGS. 10 and 11, there is shown a valve base 170 according to an alternative embodiment. Components that are similar to those provided in above-described valve base 70 have been given the same reference numbers. Base 170 is comprised of a frame that includes a plurality of C-shaped frame sections 174A, 174B, 174C and a plurality of joining elements 182 that connect together frame sections 174A, 174B and 174C. Frame sections 174A, 174B and 174C joined together by joining elements 182 define a generally cylindrical opening 194. The plurality of joining elements 182 allow adjacent frame sections 174A, 174B and 174C to move towards or away from each other in order to modify the diameter of opening 194.

In this embodiment of the present invention, joining elements 182 take the form of an expandable mesh body 184 having attachment portions 188 at opposite ends thereof. Mesh body 184 is comprised of intersecting members that intersect at intersection points. Each attachment portion 188 is fixed to respective arcuate portions 86 and 88 of adjacent frame sections, as best seen in FIG. 10. In the illustrated embodiment, attachment portions 188 are T-shaped members that are fixed within arcuate portions 86 and 88 of the frame sections. For example, arcuate portions 86, 88 may be molded around attachment portions 188. Mesh body 184 may be formed of a mesh material that is similar to that used in balloon-expandable intra vascular stents. The mesh material can be fabricated by laser-cutting or machining “windows” in a solid thin-walled tube of the appropriate metal. Cobalt-chromium is one such suitable ductile metal.

Mesh body 184 is moveable from a collapsed position (FIG. 10) to an expanded position (FIG. 11) by applying a force to frame sections 174A, 174B, 174C to move adjacent frame sections away from each other, thereby increasing the diameter of opening 194 of base 170. Such force can be applied by inflating a balloon located inside opening 194 of collapsed valve base 170, thus expanding valve base 170 to its larger, expanded shape. When fully expanded, mesh body 184 locks by way of its final dimension and the deformed shape of the mesh.

As discussed above, a medical grade fabric cover (e.g., Dacron®) covers the components of valve member 10 and base 70, 170. It should be appreciated that the cloth is stretchable to adapt to the expanded configuration of base 70, 170. Therefore, the stretchable cloth completes a seal between each of the frame sections that comprise the frame of the base 70, 170.

Bases 70, 170 may be upsized (i.e., moved from a collapsed position to an expanded position) in a variety of ways. In this regard, a specially-adapted tool or a balloon-based device may be used to facilitate moving adjacent frame sections way from each other, thereby increasing the diameter of cylindrical opening 94, 194.

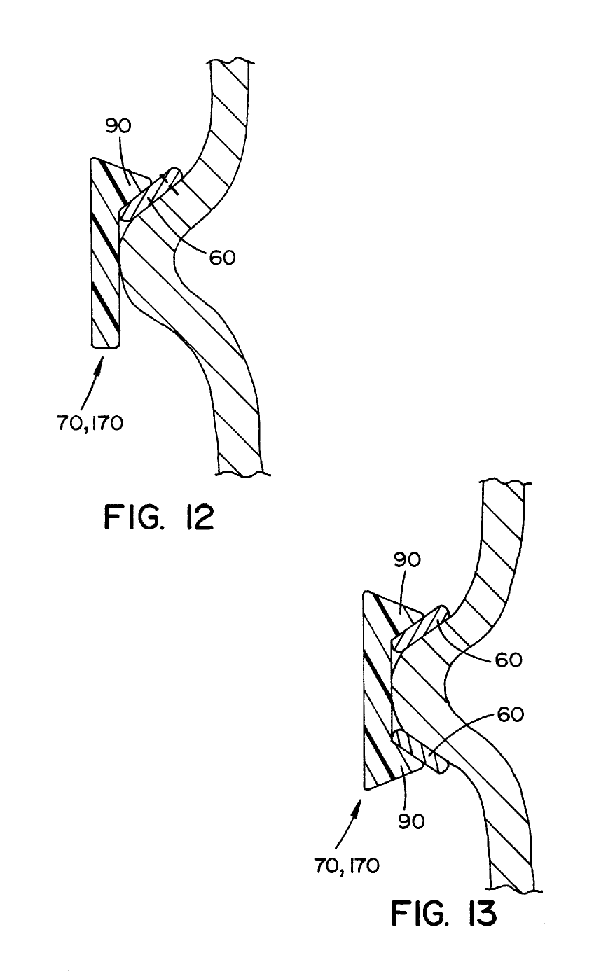

FIGS. 12-15 illustrate alternative ways to install valve bases 70 and 170. FIG. 12 shows installation of the base within an aortic annulus by sutures attaching sewing cuff 60 to the aortic annulus, as described above. FIGS. 13-15 illustrate “self-anchoring” or “sutureless” installations of the valve base. The valve base may be anchored within the aortic annulus and held therein by friction, without the use of sutures. The expandable nature of the valve base facilitates the expansion and anchoring of the valve base within the aortic annulus. According to this embodiment, the valve base cannot escape from the installed position under normal cardiac hemodynamic forces.

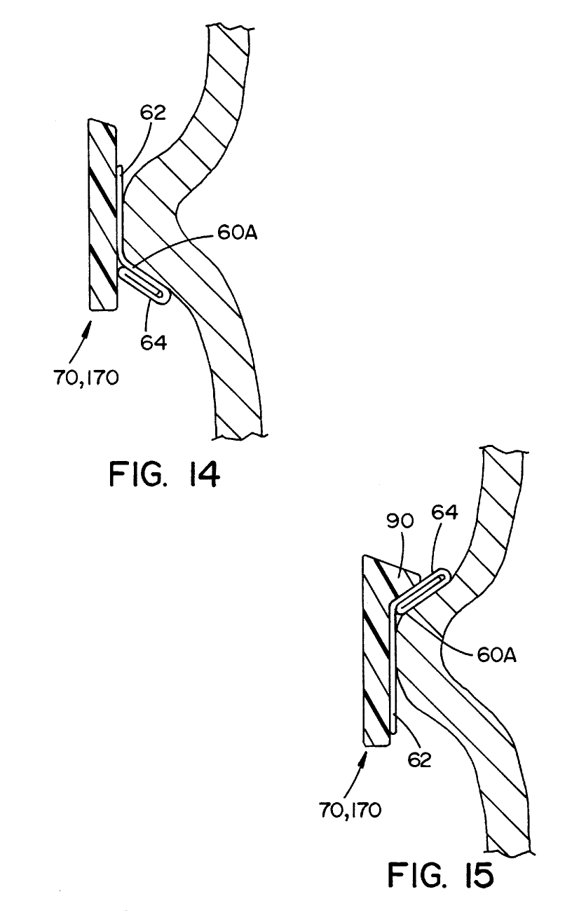

In FIG. 13, the frame sections of the base are modified to include both upper and lower flanges 90. An upper sewing cuff 60 is attached to the base such that at least a portion of the upper sewing cuff 60 is located adjacent to upper flange 90, while a lower sewing cuff 60 is attached to the base such that at least a portion of the lower sewing cuff 60 is located adjacent to lower flange 90. The upper and lower sewing cuffs 60 are not sutured to the aortic annulus for installation of the base, but rather are dimensioned and spaced to capture the aortic annulus therebetween as the base is expanded within the aortic annulus. In this respect, upper sewing cuff 60 is located above the aortic annulus and lower sewing cuff 60 is located below the aortic annulus In FIG. 14, the frame sections are modified to omit upper and lower flanges 90. A sewing cuff 60A includes a sleeve section 62 and a folded section 64 that forms a reinforced ring. When the valve base is installed, sleeve section 62 is located within the aortic annulus and folded section 64 is located below the aortic annulus. In FIG. 15, the frame sections include an upper flange 90. When the base is installed, sleeve section 62 is located within the aortic annulus and folded section 64 is located above the aortic annulus.

When first implanted into a young patient, base 70, 170 is arranged in a collapsed position (FIGS. 6 and 10). It should be appreciated that when base 70, 170 is arranged in a collapsed position, it may not have a circular shape, but instead have a tri-lobal shape, as best seen in FIG. 6. The tri-lobal shape in a collapsed position allows the base 70, 170 to have a circular shape in an expanded position. Therefore, when base 70, 170 is in a collapsed position, a non-circular shaped valve member 10 may be coupled thereto. When the base 70, 170 is in an expanded position, a circular shaped valve member 10 can be coupled thereto.

It is anticipated that the present invention could be used in the following manner. A base 70, 170 in a collapsed position (e.g., 17 mm diameter) is implanted into a young, teenage patient, and several years later when the leaflet set 11 of valve member 10 wears out, the original valve member 10 is decoupled from the implanted base 70, 170; the implanted base 70, 170 is upsized to the expanded position (e.g., 21 mm diameter); and a new, larger, adult-size valve member 10 (with a new leaflet set 11) is detachably coupled to the expanded base 70, 170.

While the collapsed (e.g., 17 mm diameter) base 70, 170 may be used with a non-circular valve member 10, it is recognized that leaflet set 11 is likely to wear out in several years (approximately 5 years) from calcification, regardless of the shape of the valve member 10, since young patients calcify valves readily. The expanded (e.g., 21 mm diameter) base 70, 170 can be used with a circular replacement valve member 10 having greater durability. The replacement valve member 10 may be subsequently replaced later in life.

The resizable valve base of the present invention also provides advantages with respect to fitting a patient with a properly sized valve assembly. As described above, proper valve size has been determined by first inserting several sizers into the patient's annulus and, by trial and error, determining which sizer fits best. Thereafter, a valve of the determined size is then implanted into the patient.

Furthermore, the resizable valve base of the present invention does not require precise sizing. In this regard, the resizable valve base of the present invention is first inserted into the patient in a collapsed state and thereafter expanded (i.e., dilated) until resistance is felt. The resizable valve base is not expanded any further, as the current size is determined to be the final size of the resizable valve base. An appropriately sized valve member with leaflet set is thereafter coupled to the installed valve base. This approach to installation has the advantage of being able to slightly distend the annulus with the valve base and thus implant the largest valve size possible.

The foregoing description are specific embodiments of the present invention. It should be appreciated that these embodiment are described for purposes of illustration only, and that numerous alterations and modifications may be practiced by those skilled in the art without departing from the spirit and scope of the invention. For example, the number of frame sections and joining elements comprising the valve base may be fewer or greater than the number shown in the illustrated embodiments. It is intended that all such modifications and alterations be included insofar as they come within the scope of the invention as claimed or the equivalents thereof.

Having described the invention, the following is claimed:

1. A resizable valve base for coupling with a valve member having a valve leaflet set, the valve base comprising:
a frame comprised of a plurality of frame sections, wherein each frame section includes a mounting portion and a pair of arcuate portions extending from opposite sides of the mounting portion; and
a plurality of joining elements for joining together the frame sections to define a generally cylindrical opening having a diameter, wherein said plurality of joining elements allow adjacent frame sections to move towards or away from each other in order to modify the diameter of the opening, thereby moving the valve base between a collapsed position and an expanded position.

2. A resizable valve base according to claim 1, wherein each frame section includes a channel for receiving one end of a joining element, said channel dimensioned to allow said one end of the joining element to move within said channel.

3. A resizable valve base according to claim 2, wherein each frame section includes a slot in communication with said channel, said one end of the joining element extending through said slot.

4. A resizable valve base according to claim 3, wherein said channel and said slot define a wall that prevents said one end of the joining element from separating from said frame section.

5. A resizable valve base according to claim 4, wherein said joining element is a band having a flange portion at opposite ends thereof, said wall dimensioned to capture the flange portion to prevent separation of the band from the frame portion.

6. A resizable valve base according to claim 5, wherein said flange portion is friction fit within said channel.

7. A resizable valve base according to claim 5, wherein said band includes a locking member to prevent the valve base from returning to a collapsed position after expansion.

8. A resizable valve base according to claim 1, wherein said plurality of joining elements are expandable mesh bodies having attachment portions at opposite ends thereof, each attachment portion fixed to respective adjacent frame sections.

9. A resizable valve base according to claim 1, wherein each said attachment portion is a T-shaped member that is fixed within said frame sections.

10. A resizable valve base according to claim 1, wherein said mounting portion is adapted to detachably couple with said valve member.

11. A resizable valve base according to claim 1, wherein said valve base has a tri-lobal shape in the collapsed position and a circular shape in the expanded position.

12. A resizable valve base according to claim 1, wherein each joining element is movable relative to a first frame section and fixed relative to a second frame section.

13. A resizable valve base according to claim 1, wherein first and second sewing cuffs are attached to the frame, said first sewing cuff located above the aortic annulus and the second sewing cuff located below the aortic annulus, when the valve base is installed.

14. A resizable valve base according to claim 1, wherein a sewing cuff is attached to the frame, said sewing cuff having sections located within and above the aortic annulus when the valve base is installed.

15. A resizable valve base according to claim 1, wherein a sewing cuff is attached to the frame, said sewing cuff having sections located within and below the aortic annulus when the valve base is installed.
